(19) United States
(12) Patent Application Publication
(10) Pub. No.: US 2020/0022430 A1
(43) Pub. Date: Jan. 23, 2020

(54) Phototherapy Anti-Smog Cloth Structure

(71) Applicant: Inprotex Co., Ltd., Taipei (TW)

(72) Inventor: Man-Ching Wang, Taipei (TW)

(21) Appl. No.: 16/038,315

(22) Filed: Jul. 18, 2018

(51) Int. Cl. A41D 13/11; A41D 27/10; D03D 1/00; D03D 11/00; D06C 7/00; B01D 39/08; A62B 18/02; A62B 7/10; A61N 5/06

(52) U.S. Cl. CPC A41D 13/11; A47L 9/14; D03D 1/007; D03D 11/00; D06C 7/00; B01D 39/083; A62B 18/025; A62B 7/10; A61N 5/0613; A41D 2400/26; A41D 2400/28; A41D 2400/32; D03D 2700/0103; D03D 2700/0111; D10B 2331/02; D10B 2401/061; D10B 2401/22; D10B 2501/04; D03D 2700/0133; A41D 27/10

(57) ABSTRACT

A cloth structure includes a first weaving layer, a second weaving layer interwoven with the first weaving layer, an intermediate connecting layer located between the first weaving layer and the second weaving layer, and a sun protection layer applied on the first weaving layer. The cloth structure presents a single-cloth fabric. The sun protection layer covers pores of the first weaving layer and forms a first protection layer to filter the smog. The first weaving layer is formed with inverted V-shaped cloth patterns, so that the first weaving layer forms a second protection layer to filter the smog. The intermediate connecting layer forms a third protection layer to filter the smog. The second weaving layer obstructs the thread openings of the first weaving layer and forms a fourth protection layer to filter the smog.

### BACKGROUND OF THE INVENTION

### 1. Field of the Invention

The present invention relates to a cloth structure and, more particularly, to a phototherapy anti-smog cloth structure.

### 2. Description of the Related Art

A conventional sun protection light cloth structure 10 (the applicant's Taiwanese Patent No. 1400376) in accordance with the prior art shown in FIGS. 10 and 11 comprises a bottom cloth layer and a color layer 101 combined with the bottom cloth layer. The method for forming the conventional cloth structure comprises providing staples including a polyester fiber (such as Nylon) and a resilient yarn (such as SpanDEX), weaving the staples to form a fabric, processing the fabric at a high temperature to form a stereotype cloth of the bottom cloth layer, treating the stereotype cloth by a non-toxic fluorescent bleaching process to form the bottom cloth layer, applying a fluorescent color paste on the bottom cloth layer to form the color layer 101 in a tension-free full-out layering coloring manner, drying the bottom cloth layer by a dryer in a tension-free and dust-free manner, and removing formaldehyde from the bottom cloth layer at a high temperature, to form the final product of the cloth structure 10. However, the color layer 101 is not attached to the bottom cloth layer solidly and stably, so that the color layer 101 is easily detached from the bottom cloth layer due to frequent washing during a long-term utilization, thereby failing the color layer 101, and thereby decreasing the function of the cloth structure. In addition, the conventional sun protection light cloth structure cannot isolate and filter the smog.

A conventional anti-smog product 20 in accordance with the prior art shown in FIG. 12 comprises an inner cloth layer 21, an outer cloth layer 22, and multiple isolating layers 23 located between the inner cloth layer 21 and the outer cloth layer 22. Each of the isolating layers 23 is made of non-woven fabric or active carbon to provide an anti-smog function. However, the isolating layers 23 are not air ventilating so that the wearer easily sweats and feels uncomfortable when wearing the conventional anti-smog product 20 during a period of time. Thus, the user cannot wear the conventional anti-smog product 20 very long.

### BRIEF SUMMARY OF THE INVENTION

In accordance with the present invention, there is provided a cloth structure comprising a first weaving layer, a second weaving layer, an intermediate connecting layer located between the first weaving layer and the second weaving layer, and a sun protection layer mounted on the first weaving layer. A method for forming the cloth structure comprises a first step of preparing material, a second step of weaving, a third step of shaping cloth, a fourth step of dyeing and finishing, a fifth step of tension-free full-out layering coloring, a sixth step of tension-free and dust-free drying, and a seventh step of shaping and dispersing. The first step includes providing a first texture material polymer, a second texture material polymer and a third texture material polymer. The first texture material polymer is made of polyester fiber nylon and elastic yarn. The second texture material polymer is made of polyester fiber and elastic yarn. The third texture material polymer is made of polyester fiber. The third texture material polymer is processed by fine grinding. The second step includes performing double-sided weaving to weave the first texture material polymer for forming the first weaving layer, to weave the second texture material polymer for forming the second weaving layer, and to weave the third texture material polymer for forming the intermediate connecting layer, with the intermediate connecting layer being located between the first weaving layer and the second weaving layer, to form a single-cloth fabric which includes the first weaving layer, the second weaving layer and the intermediate connecting layer. The intermediate connecting layer is interwoven between the first weaving layer and the second weaving layer simultaneously when the first weaving layer and the second weaving layer are woven. The third step includes treating the single-cloth fabric at a temperature of 180° C.±10° C., to integrally combine the first texture material polymer, the second texture material polymer and the third texture material polymer, to construct a shaped grey. The fourth step includes dyeing and finishing the first weaving layer. The fifth step includes applying a non-toxic fluorescent color paste on the first weaving layer to form the sun protection layer on a surface of the first weaving layer. The sixth step includes drying the first weaving layer in a dryer by tension-free and dust-free drying. The seventh step includes shaping and dispersing the single-cloth fabric at a high temperature of 170-180° C., to remove formaldehyde from the single-cloth fabric.

Further benefits and advantages of the present invention will become apparent after a careful reading of the detailed description with appropriate reference to the accompanying drawings.

### BRIEF DESCRIPTION OF THE SEVERAL VIEWS OF THE DRAWING(S)

FIG. 1 is a flow chart of a method for manufacturing a cloth structure in accordance with the preferred embodiment of the present invention.

FIG. 2 is a perspective view of a cloth structure in accordance with the preferred embodiment of the present invention.

FIG. 3 is a partially side cross-sectional view of the cloth structure in accordance with the preferred embodiment of the present invention.

FIG. 4 is a front cross-sectional view of the cloth structure in accordance with the preferred embodiment of the present invention.

FIG. 5 is a locally enlarged cross-sectional operational view showing that the cloth structure isolates the smog particles.

FIG. 6 is a perspective view showing the cloth structure for a medical mask.

FIG. 7 is a perspective view showing the cloth structure for a neck cover.

FIG. 8 is a perspective view showing the cloth structure for a sleevelet.

FIG. 9 is a perspective view showing the cloth structure for a dust bag of a vacuum cleaner.

FIG. 10 is a flow chart of a method for manufacturing a conventional cloth structure in accordance with the prior art.

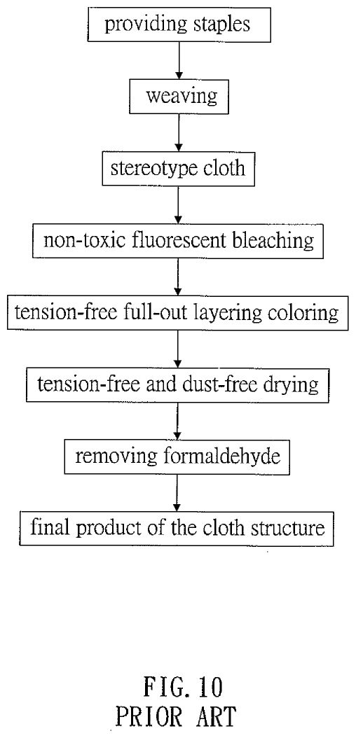

FIG. 11 is a perspective view of a conventional cloth structure in accordance with the prior art.

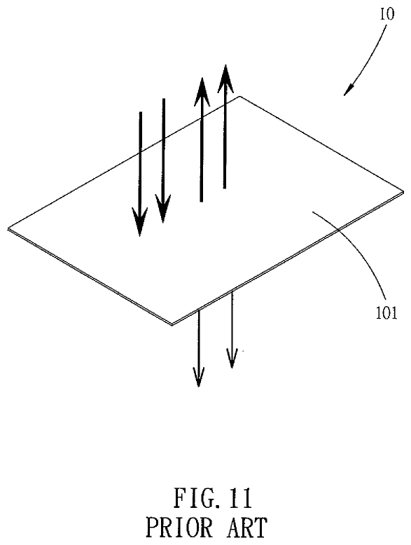

FIG. 12 is a partially side cross-sectional view of another conventional cloth structure in accordance with the prior art.

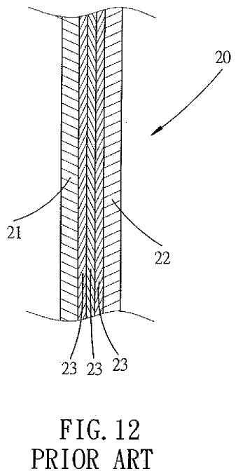

### DETAILED DESCRIPTION OF THE INVENTION

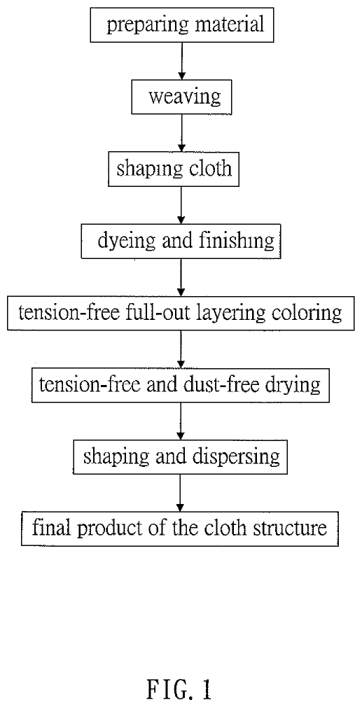

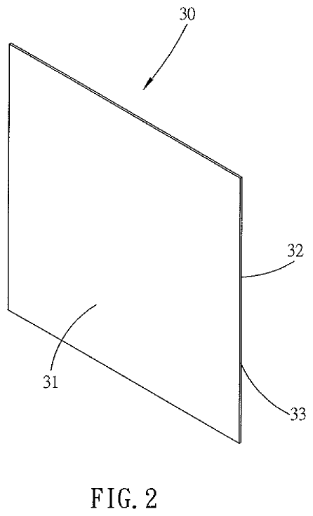

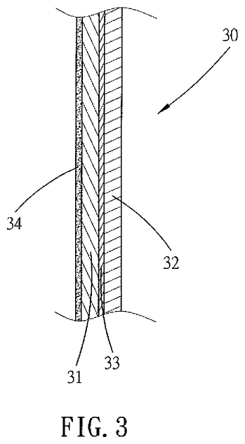

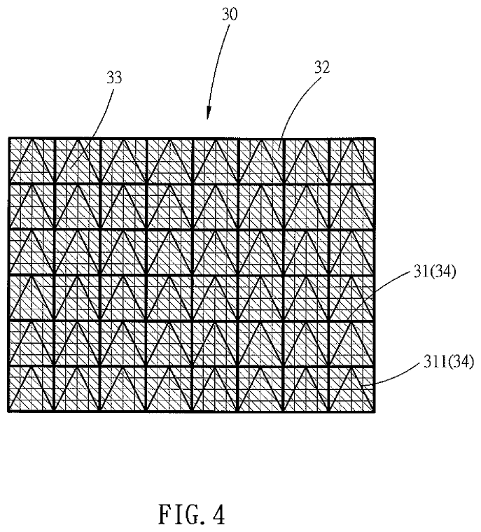

Referring to the drawings and initially to FIGS. 1-5, a cloth structure 30 in accordance with the preferred embodiment of the present invention comprises a first weaving layer 31, a second weaving layer 32, an intermediate connecting layer 33 located between the first weaving layer 31 and the second weaving layer 32, and a sun protection layer 34 mounted on the first weaving layer 31.

A method for forming the cloth structure 30 comprises a first step (A) of preparing material, a second step (B) of weaving, a third step (C) of shaping cloth, a fourth step (D) of dyeing and finishing, a fifth step (E) of tension-free full-out layering coloring, a sixth step (F) of tension-free and dust-free drying, and a seventh step (G) of shaping and dispersing.

The first step (A) includes providing a first texture material polymer, a second texture material polymer and a third texture material polymer. The first texture material polymer is made of polyester fiber nylon and elastic yarn. The second texture material polymer is made of polyester fiber and elastic yarn. The third texture material polymer is made of polyester fiber. The third texture material polymer is processed by fine grinding.

The second step (B) includes performing double-sided weaving to weave the first texture material polymer for forming the first weaving layer 31, to weave the second texture material polymer for forming the second weaving layer 32, and to weave the third texture material polymer for forming the intermediate connecting layer 33, with the intermediate connecting layer 33 being located between the first weaving layer 31 and the second weaving layer 32, to form a single-cloth fabric which includes the first weaving layer 31, the second weaving layer 32 and the intermediate connecting layer 33. The intermediate connecting layer 33 is interwoven between the first weaving layer 31 and the second weaving layer 32 simultaneously when the first weaving layer 31 and the second weaving layer 32 are woven. The single-cloth fabric is elastic, ventilating and thin and has a light weight.

The third step (C) includes clearing impurities contained in the elastic yarn of the single-cloth fabric, controlling a stitch density of the single-cloth fabric, and treating the single-cloth fabric at a temperature of 180° C±10° C., to integrally combine the first texture material polymer, the second texture material polymer and the third texture material polymer, to construct a shaped grey.

The fourth step (D) includes dyeing and finishing the first weaving layer 31.

The fifth step (E) includes applying a non-toxic fluorescent color paste on the first weaving layer 31 to form the sun protection layer 34 on a surface of the first weaving layer 31. Preferably, the non-toxic fluorescent color paste is applied on the first weaving layer 31 by a two-axle roller and by single-face tension-free full-out layering coloring, so that the non-toxic fluorescent color paste is distributed evenly on the first weaving layer 31. In addition, the molecules of the non-toxic fluorescent color paste (or compound) have a high light-absorbing capacity to convert harmful ultraviolet rays into beneficial visible rays, including red, orange, yellow, green, blue, indigo and purple rays, to promote the blood circulation of the wearer. Thus, the molecules of the non-toxic fluorescent compound absorb the solar energy of the solar rays (with a wavelength of 780nm-390nm), to exactly regulate and convert the solar rays of various colors, including red, orange, yellow, green, blue, indigo and purple, that are beneficial to the human body.

The sixth step (F) includes drying the first weaving layer 31 in a dryer by tension-free and dust-free drying to enhance the drying effect, thereby preventing the elasticity and light permeability of the first weaving layer 31 from being decreased due to an excessive pulling force applied on the first weaving layer 31, and thereby preventing the formaldehyde, dust and impurities from remaining on and contaminating the first weaving layer 31.

The seventh step (G) includes shaping and dispersing the single-cloth fabric at a high temperature of 170-180° C., to remove the formaldehyde from the single-cloth fabric, to produce a final product of the cloth structure 30. The single-cloth fabric is shaped and dispersed at the high temperature of 170-180° C., to clear and prevent the formaldehyde from remaining in the single-cloth fabric, to distribute the non-toxic fluorescent color paste more evenly, and to enhance the color fastness.

In the preferred embodiment of the present invention, the sun protection layer 34 is dried in the dryer by tension-free and dust-free drying, and is shaped and dispersed at the high temperature of 170-180° C., so that the sun protection layer 34 covers pores of the first weaving layer 31. Thus, the sun protection layer 34 forms a first protection layer to filter the smog.

In the preferred embodiment of the present invention, the polyester fiber nylon and the elastic yarn of the first texture material polymer are interwoven by high-weave weaving to form the first weaving layer 31, so that the first weaving layer 31 has an enhanced cloth density. Preferably, the polyester fiber nylon and the elastic yarn of the first texture material polymer are interwoven by a needle counting machine to form a plurality of substantially inverted V-shaped cloth patterns 311 in the first weaving layer 31, to obstruct thread openings of the first weaving layer 31. Thus, the first weaving layer 31 forms a second protection layer to filter the smog. In practice, the polyester fiber nylon of the first weaving layer 31 provides a cooling and soft sensation and has a deodorizing feature. Besides, the polyester fiber nylon of the first weaving layer 31 has a high light-absorbing capacity, so that the first weaving layer 31 is dyed and colored easily at a low temperature of 98° C., and the non-toxic fluorescent color paste is applied on the first weaving layer 31 easily.

In the preferred embodiment of the present invention, the polyester fiber of the intermediate connecting layer 33 has a hydrophobic feature and produces a micro static electricity and an attractive force (in a dry environment) to attract the smog. Thus, the intermediate connecting layer 33 forms a third protection layer to filter the smog.

In the preferred embodiment of the present invention, the polyester fiber of the second weaving layer 32 has a thread count greater than that of the polyester fiber nylon of the first weaving layer 31. Preferably, the thread count of the polyester fiber of the second weaving layer 32 is smaller than 3.5 times of that of the polyester fiber nylon of the first weaving layer 31. Thus, the second weaving layer 32 obstructs the thread openings of the first weaving layer 31 and forms a fourth protection layer to filter the smog.

In the preferred embodiment of the present invention, the fourth step (D) further includes dyeing and finishing the second weaving layer 32 according to a customized requirement. Preferably, the second weaving layer 32 is dyed and finished to have a color different that of the first weaving layer 31.

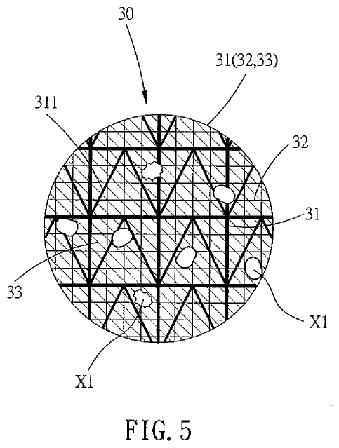

In conclusion, the cloth structure 30 in accordance with the preferred embodiment of the present invention presents a single-cloth fabric and comprises a first weaving layer 31 functioning as a front face, a second weaving layer 32 functioning as a back face and interwoven with the first weaving layer 31, an intermediate connecting layer 33 functioning as a middle section and located between the first weaving layer 31 and the second weaving layer 32, and a sun protection layer 34 applied on a surface of the first weaving layer 31. The sun protection layer 34 is processed by a non-toxic fluorescent coloring technology, processed by single-face tension-free full-out layering coloring, dried in a dryer by tension-free and dust-free drying, and is shaped and dispersed at a high temperature to clear the formaldehyde. The sun protection layer 34 covers pores of the first weaving layer 31 and forms a first protection layer to filter the smog. The first weaving layer 31 is formed by a first texture material polymer which is made of polyester fiber nylon and elastic yarn. The second weaving layer 32 is formed by a second texture material polymer which is made of polyester fiber and elastic yarn. The intermediate connecting layer 33 is formed by a third texture material polymer which is made of polyester fiber. The polyester fiber nylon and the elastic yarn of the first texture material polymer are interwoven by high-weave weaving to form the first weaving layer 31, with the first weaving layer 31 having an enhanced cloth density. The first weaving layer 31 is formed with a plurality of substantially inverted V-shaped cloth patterns 311 which obstruct thread openings of the first weaving layer 31, with the first weaving layer 31 forming a second protection layer to filter the smog. The polyester fiber of the intermediate connecting layer 33 has a hydrophobic feature and produces a micro static electricity and an attractive force to attract the smog, with the intermediate connecting layer 33 forming a third protection layer to filter the smog. The polyester fiber of the second weaving layer 32 has a thread count smaller than 3.5 times of that of the polyester fiber nylon of the first weaving layer 31, with the second weaving layer 32 obstructing the thread openings of the first weaving layer 31 and forming a fourth protection layer to filter the smog. Thus, the cloth structure 30 constructs four protection layers to filter the smog particles “X1” of different sizes as shown in FIG. 5.

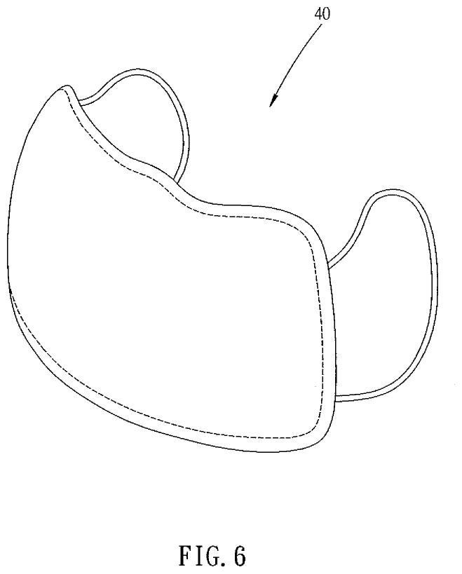

Accordingly, the cloth structure 30 constructs multiple protection layers by provision of the first weaving layer 31, the second weaving layer 32, the intermediate connecting layer 33 and the sun protection layer 34, so as to filter the smog efficiently. In addition, the cloth structure 30 has a high light-absorbing capacity to convert harmful ultraviolet (UV) rays into beneficial visible rays, so that the cloth structure 30 an anti-UV function, so as to provide a phototherapy effect. Further, the cloth structure 30 is air ventilating and light permeable and is cleaned and washed repeatedly. Further, the cloth structure 30 has a sun protection function by provision of the sun protection layer 34. Further, the stitch and cloth density of the cloth structure 30 is controlled by a weaving technology, so as to achieve an anti-smog function, without having to paint any anti-smog agent. As shown in FIG. 6, the cloth structure is worked and is worn by a user to filter the smog, and to provide sun protection and phototherapy. Preferably, the cloth structure is available for a medical mask 40.

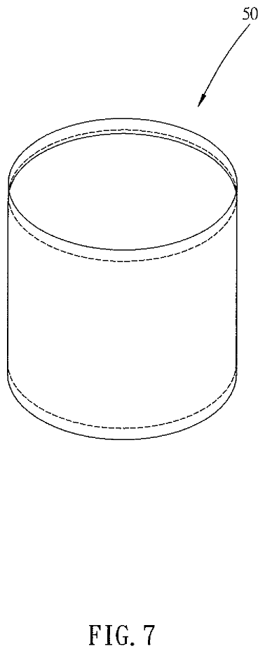

As shown in FIG. 7, the cloth structure is available for a neck cover 50.

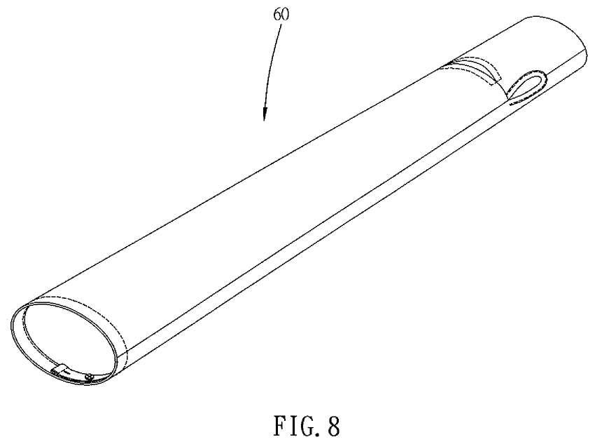

As shown in FIG. 8, the cloth structure is available for a sleevelet 60. Alternatively, the cloth structure is available for a clothing

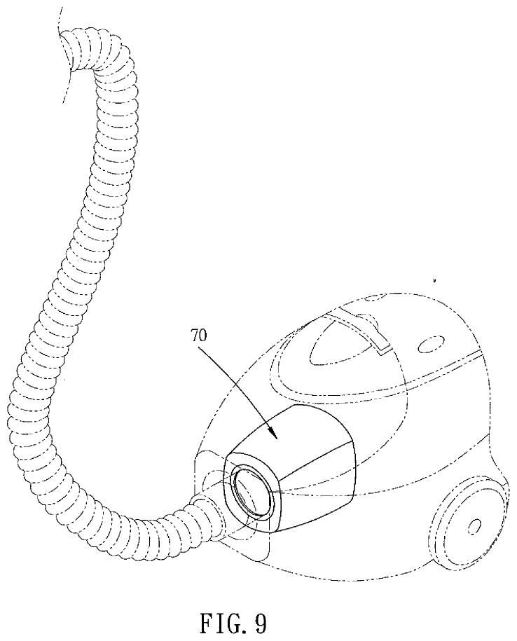

As shown in FIG. 9, the cloth structure is available for a dust bag 70 of a vacuum cleaner.

It is appreciated that, the cloth structure is elastic, ventilating and thin, and has a light weight, to provide a comfortable sensation the wearer. In addition, the cloth structure isolates and converts the harmful ultraviolet rays into beneficial visible rays which are projected on the wearer's skin, so as to achieve an anti-UV function and to provide a phototherapy function to the wearer. Further, the cloth structure provides multiple protection layers so as to filter the smog efficiently. Further, the cloth structure has a sun protection function.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the scope of the invention.

1. A cloth structure comprising:
a first weaving layer;
a second weaving layer;
an intermediate connecting layer located between the first weaving layer and the second weaving layer; and
a sun protection layer mounted on the first weaving layer;
wherein:
a method for forming the cloth structure comprises a first step of preparing material, a second step of weaving, a third step of shaping cloth, a fourth step of dyeing and finishing, a fifth step of tension-free full-out layering coloring, a sixth step of tension-free and dust-free drying, and a seventh step of shaping and dispersing;
the first step includes providing a first texture material polymer, a second texture material polymer and a third texture material polymer;
the first texture material polymer is made of polyester fiber nylon and elastic yarn;
the second texture material polymer is made of polyester fiber and elastic yarn;
the third texture material polymer is made of polyester fiber;
the third texture material polymer is processed by fine grinding;
the second step includes performing double-sided weaving to weave the first texture material polymer for forming the first weaving layer, to weave the second texture material polymer for forming the second weaving layer, and to weave the third texture material polymer for forming the intermediate connecting layer, with the intermediate connecting layer being located between the first weaving layer and the second weaving layer, to form a single-cloth fabric which includes the first weaving layer, the second weaving layer and the intermediate connecting layer;
the intermediate connecting layer is interwoven between the first weaving layer and the second weaving layer simultaneously when the first weaving layer and the second weaving layer are woven;
the third step includes treating the single-cloth fabric at a temperature of 180° C.±10° C., to integrally combine the first texture material polymer, the second texture material polymer and the third texture material polymer, to construct a shaped grey;
the fourth step includes dyeing and finishing the first weaving layer;
the fifth step includes applying a non-toxic fluorescent color paste on the first weaving layer to form the sun protection layer on a surface of the first weaving layer;
the sixth step includes drying the first weaving layer in a dryer by tension-free and dust-free drying; and
the seventh step includes shaping and dispersing the single-cloth fabric at a high temperature of 170-180° C., to remove formaldehyde from the single-cloth fabric.

2. The cloth structure of claim 1, wherein:
the non-toxic fluorescent color paste is applied on the first weaving layer by a two-axle roller and by single-face tension-free full-out layering coloring, so that the non-toxic fluorescent color paste is distributed evenly on the first weaving layer; and
the sun protection layer is dried in the dryer by tension-free and dust-free drying, and is shaped and dispersed at the high temperature of 170-180° C., so that the sun protection layer covers pores of the first weaving layer and forms a first protection layer to filter smog.

3. The cloth structure of claim 1, wherein:
the polyester fiber nylon and the elastic yarn of the first texture material polymer are interwoven by high-weave weaving to form the first weaving layer;
the polyester fiber nylon and the elastic yarn of the first texture material polymer are interwoven by a needle counting machine to form a plurality of substantially inverted V-shaped cloth patterns in the first weaving layer, to obstruct thread openings of the first weaving layer; and
the first weaving layer forms a second protection layer to filter the smog.

4. The cloth structure of claim 1, wherein:
the polyester fiber of the intermediate connecting layer has a hydrophobic feature and produces a micro static electricity and an attractive force to attract the smog; and
the intermediate connecting layer forms a third protection layer to filter the smog.

5. The cloth structure of claim 1, wherein:
the polyester fiber of the second weaving layer has a thread count greater than that of the polyester fiber nylon of the first weaving layer;
the thread count of the polyester fiber of the second weaving layer is smaller than 3.5 times of that of the polyester fiber nylon of the first weaving layer; and
the second weaving layer obstructs the thread openings of the first weaving layer and forms a fourth protection layer to filter the smog.

6. The cloth structure of claim 1, wherein the fourth step further includes dyeing and finishing the second weaving layer according to a customized requirement, and the second weaving layer is dyed and finished to have a color different that of the first weaving layer.

7. The cloth structure of claim 1, wherein the cloth structure is worked and is worn by a user to filter the smog, and to provide sun protection and phototherapy.

8. The cloth structure of claim 7, wherein the cloth structure is available for a medical mask, a neck cover, a sleevelet, a clothing or a dust bag of a vacuum cleaner.

9. A cloth structure comprising:
a first weaving layer functioning as a front face;
a second weaving layer functioning as a back face and interwoven with the first weaving layer;
an intermediate connecting layer functioning as a middle section and located between the first weaving layer and the second weaving layer; and
a sun protection layer applied on a surface of the first weaving layer;
wherein:
the cloth structure presents a single-cloth fabric;
the sun protection layer is processed by a non-toxic fluorescent coloring technology, processed by single-face tension-free full-out layering coloring, dried in a dryer by tension-free and dust-free drying, and is shaped and dispersed at a high temperature to clear the formaldehyde;
the sun protection layer covers pores of the first weaving layer and forms a first protection layer to filter the smog;
the first weaving layer is formed by a first texture material polymer which is made of polyester fiber nylon and elastic yarn;
the second weaving layer is formed by a second texture material polymer which is made of polyester fiber and elastic yarn;
the intermediate connecting layer is formed by a third texture material polymer which is made of polyester fiber;
the polyester fiber nylon and the elastic yarn of the first texture material polymer are interwoven by high-weave weaving to form the first weaving layer;
the first weaving layer is formed with a plurality of substantially inverted V-shaped cloth patterns which obstruct thread openings of the first weaving layer, with the first weaving layer forming a second protection layer to filter the smog;
the polyester fiber of the intermediate connecting layer has a hydrophobic feature and produces a micro static electricity and an attractive force to attract the smog, with the intermediate connecting layer forming a third protection layer to filter the smog; and
the polyester fiber of the second weaving layer has a thread count smaller than 3.5 times of that of the polyester fiber nylon of the first weaving layer, with the second weaving layer obstructing the thread openings of the first weaving layer and forming a fourth protection layer to filter the smog.
